(19) United States
(12) Patent Application Publication
(10) Pub. No.: US 2026/0311805 A1
(43) Pub. Date: Oct. 8, 2026

(54) SYSTEM AND METHOD FOR PREDICTING PHENOTYPES

(71) Applicant: LG MANAGEMENT DEVELOPMENT INSTITUTE CO., LTD., Seoul (KR)

(72) Inventors: Ki Young KIM, Seoul (KR); Do Yeong HWANG, Seoul (KR); Soo Rin YIM, Seoul (KR); Sung Joon PARK, Seoul (KR); Kyung Wook LEE, Seoul (KR)

(21) Appl. No.: 19/462,640

(22) Filed: Jan. 28, 2026

Related U.S. Application Data
(63) Continuation of application No. PCT/KR2025/013746, filed on Sep. 5, 2025.

(30) Foreign Application Priority Data
Feb. 26, 2025 (KR) 10-2025-0025257
Jun. 12, 2025 (KR) 10-2025-0077367

(51) Int. Cl. G16B 20/40; G06N 3/047; G16B 40/20; G16H 50/20

(52) U.S. Cl. CPC G16B 20/40; G06N 3/047; G16B 40/20; G16H 50/20

(57) ABSTRACT

The system for predicting phenotypes includes: at least one memory configured to store one or more instructions; and at least one processor configured to execute the one or more instructions stored in the at least one memory. Operations performed by the one or more instructions include learning to predict phenotypes from omics data. The learning to predict phenotypes includes: acquiring at least one dataset including omics data; generating omics features from the omics data; generating, by a reconstruction model, a latent vector from the omics features; and predicting, by a phenotype predicting model, phenotypes from the latent vector.

### CROSS REFERENCE TO RELATED APPLICATIONS

This application is a Bypass Continuation of International Patent Application No. PCT/KR2025/013746, filed on Sep. 5, 2025, which claims priority from and the benefit of Korean Patent Application Nos. 10-2025-0025257, filed on Feb. 26, 2025 and 10-2025-0077367, filed on Jun. 12, 2025, which is hereby incorporated by reference for all purposes as if fully set forth herein.

### BACKGROUND

### Field

Embodiments of the invention relate generally to a system and method for predicting phenotypes, and more particularly, the present disclosure relates to a system and method with improved performance capable of efficiently predicting phenotypes from omics data.

### Discussion of the Background

Omics data, which is generated by analyzing various biomolecules possessed by a living organism, contains various information, and by analyzing omics data, it is possible to identify the disease state or risk of the living organism as well as the biomolecules affecting the disease.

With the recent advancement of life science technology, the time and cost required to produce omics data have decreased exponentially, while the amount of available omics data has increased enormously. Accordingly, attempts have increased to learn artificial intelligence models with omics data in order to understand phenomena occurring in living organisms.

However, omics data has a vast number of variables, while most of the variables have little effect on a specific phenotype, and thus it is difficult to efficiently predict phenotypes from omics data. As such, omics data has the problems of high dimensionality and sparsity, and the complexity of the data causes various problems such as overfitting, reduced model interpretability, and computational difficulties.

The above information disclosed in this Background section is only for understanding of the background of the inventive concepts, and, therefore, it may contain information that does not constitute prior art.

### SUMMARY

The present disclosure provides a system and method capable of predicting phenotypes from omics data, and further to provide a system and method for predicting phenotypes, having excellent performance while efficiently performing prediction from vast omics data.

The present disclosure provides a system configured to predict phenotypes as a means for solving the above problem, the system configured to predict phenotypes including a step in which a phenotype predicting model predicts phenotypes from a latent vector.

Additional features of the inventive concepts will be set forth in the description which follows, and in part will be apparent from the description, or may be learned by practice of the inventive concepts.

According to an embodiment of the present disclosure, a system for predicting phenotypes includes: at least one memory configured to store one or more instructions; and at least one processor configured to execute the one or more instructions stored in the at least one memory, in which operations performed by the one or more instructions include learning to predict phenotypes from omics data, in which the learning to predict phenotypes includes: acquiring at least one dataset including omics data; generating omics features from the omics data; generating, by a reconstruction model, a latent vector from the omics features; and predicting, by a phenotype predicting model, phenotypes from the latent vector.

The reconstruction model may include a first reconstruction sub-neural network and a second reconstruction sub-neural network, the first reconstruction sub-neural network generating the latent vector from the omics features, and the second reconstruction sub-neural network generating reconstructed omics features from the latent vector, in which the learning to predict phenotypes includes: generating, by the first reconstruction sub-neural network, the latent vector from the omics features; generating, by the second reconstruction sub-neural network, the reconstructed omics features from the latent vector; comparing the reconstructed omics features with the omics features to evaluate performance of the reconstruction model; and learning the reconstruction model based on the evaluation.

The learning to predict phenotypes may include at least one of: respectively learning the reconstruction model and the phenotype predicting model; alternately learning the reconstruction model and the phenotype predicting model; or after alternately learning the reconstruction model and the phenotype predicting model, additionally learning the phenotype predicting model.

The phenotype predicting model may further receive one or more individual features as input to predict phenotypes.

The phenotypes may be associated with a disease.

The omics data may include one or more selected from genomic data, transcriptomic data, proteomic data, metabolomic data, lipidomic data, and combinations thereof.

The system may is configured to obtain disease-associated biomarker information or disease-associated phenotype information of an individual.

According to another embodiment of the present disclosure, a method for predicting phenotypes performed by at least one processor may include: learning to predict phenotypes from omics data, wherein the learning to predict phenotypes includes: acquiring at least one dataset including omics data; generating omics features from the omics data; generating, by a reconstruction model, a latent vector from the omics features; and predicting, by a phenotype predicting model, phenotypes from the latent vector.

The reconstruction model may include a first reconstruction sub-neural network and a second reconstruction sub-neural network, the first reconstruction sub-neural network generating the latent vector from the omics features, and the second reconstruction sub-neural network generating reconstructed omics features from the latent vector, in which the learning to predict phenotypes includes: generating, by the first reconstruction sub-neural network, the latent vector from the omics features; generating, by the second reconstruction sub-neural network, the reconstructed omics features from the latent vector; comparing the reconstructed omics features with the input omics features to evaluate performance of the reconstruction model; and learning the reconstruction model based on the evaluation.

The learning to predict phenotypes may include at least one of: (1) respectively learning the reconstruction model and the phenotype predicting model; (2) alternately learning the reconstruction model and the phenotype predicting model; or (3) after alternately learning the reconstruction model and the phenotype predicting model, additionally learning the phenotype predicting model.

The phenotype predicting model may further receive one or more individual features as input to predict phenotypes.

The phenotypes may be associated with a disease.

The omics data may include one or more selected from genomic data, transcriptomic data, proteomic data, metabolomic data, lipidomic data, and combinations thereof.

The method for predicting phenotypes may further include obtaining disease-associated biomarker information or obtaining disease-associated phenotype information of an individual.

According to another embodiment of the present disclosure, a method for predicting phenotypes performed by at least one processor may include learning to predict phenotypes from omics data, wherein the learning to predict phenotypes includes: acquiring at least one dataset including omics data; generating omics features from the omics data; generating, by a reconstruction model, a latent vector from the omics features; and predicting, by a phenotype predicting model, phenotypes from the latent vector.

According to another embodiment of the present disclosure, a computerized method for predicting phenotypes may include: learning to predict phenotypes from omics data, in which the learning to predict phenotypes includes: acquiring at least one dataset including omics data; generating omics features from the omics data; generating, by a reconstruction model, a latent vector from the omics features; and predicting, by a phenotype predicting model, phenotypes from the latent vector.

The reconstruction model may include a first reconstruction sub-neural network and a second reconstruction sub-neural network, the first reconstruction sub-neural network generating the latent vector from the omics features, and the second reconstruction sub-neural network generating reconstructed omics features from the latent vector, in which the learning to predict phenotypes includes: generating, by the first reconstruction sub-neural network, the latent vector from the omics features; generating, by the second reconstruction sub-neural network, the reconstructed omics features from the latent vector; comparing the reconstructed omics features with the input omics features to evaluate performance of the reconstruction model; and learning the reconstruction model based on the evaluation.

The learning to predict phenotypes may include at least one of: (1) respectively learning the reconstruction model and the phenotype predicting model; (2) alternately learning the reconstruction model and the phenotype predicting model; or (3) after alternately learning the reconstruction model and the phenotype predicting model, additionally learning the phenotype predicting model.

The phenotype predicting model may further receives one or more individual features as input to predict phenotypes.

The phenotypes may be associated with a disease.

The omics data may include one or more selected from genomic data, transcriptomic data, proteomic data, metabolomic data, lipidomic data, and combinations thereof.

The method for predicting phenotypes may further include obtaining disease-associated biomarker information or obtaining disease-associated phenotype information of an individual.

It is to be understood that both the foregoing general description and the following detailed description are exemplary and explanatory and are intended to provide further explanation of the invention as claimed.

### BRIEF DESCRIPTION OF THE DRAWINGS

The accompanying drawings, which are included to provide a further understanding of the invention and are incorporated in and constitute a part of this specification, illustrate embodiments of the invention, and together with the description serve to explain the inventive concepts.

FIG. 1 is a schematic block diagram of an apparatus capable of executing a system and method for predicting phenotypes according to an embodiment of the present disclosure.

FIG. 2 is a schematic flowchart of a system and method for predicting phenotypes according to an embodiment of the present disclosure.

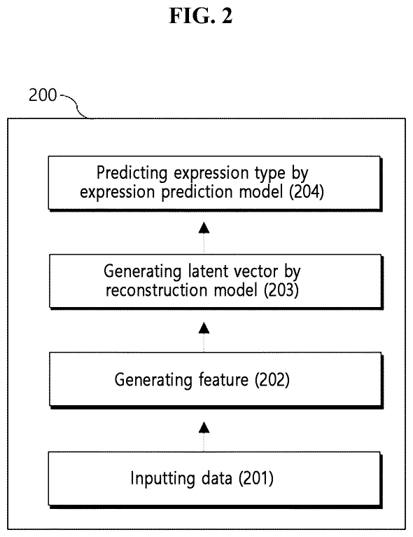

FIG. 3 is a schematic example diagram showing a method of predicting phenotypes according to a system for predicting phenotypes according to an embodiment of the present disclosure.

FIG. 4 is a schematic example diagram showing a method of learning a reconstruction model according to an embodiment of the present disclosure.

FIG. 5 is a schematic example diagram showing a method of learning a phenotype predicting model according to an embodiment of the present disclosure.

FIG. 6 is a schematic example diagram showing a method of obtaining disease-associated phenotype information of an individual by using a phenotype predicting model according to an embodiment of the present disclosure.

FIG. 7 is a schematic example diagram showing a method of obtaining disease-associated biomarker information by using a phenotype predicting model according to an embodiment of the present disclosure.

FIG. 8 is a schematic diagram showing a relationship between omics data, individual data, and phenotypes.

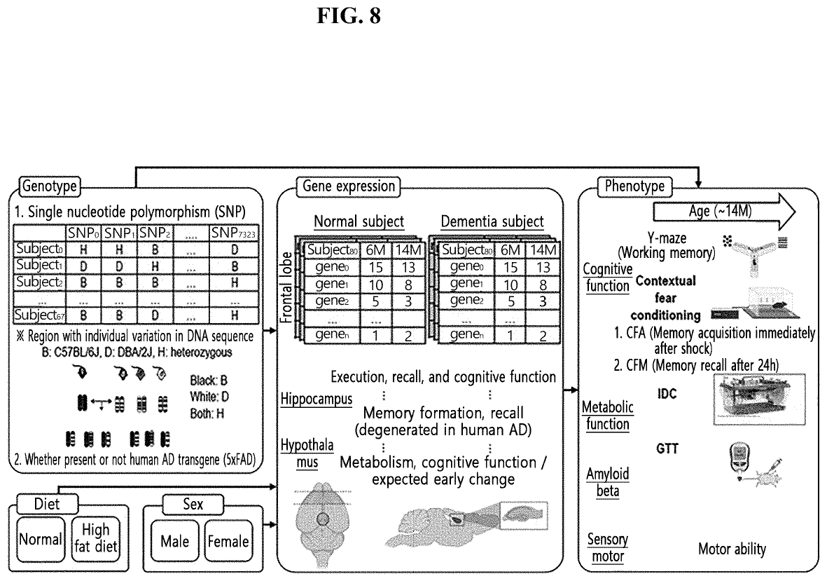

### DETAILED DESCRIPTION

In the following description, for the purposes of explanation, numerous specific details are set forth in order to provide a thorough understanding of various embodiments or implementations of the invention. As used herein “embodiments” and “implementations” are interchangeable words that are non-limiting examples of devices or methods employing one or more of the inventive concepts disclosed herein. It is apparent, however, that various embodiments may be practiced without these specific details or with one or more equivalent arrangements. In other instances, well-known structures and devices are shown in block diagram form in order to avoid unnecessarily obscuring various embodiments. Further, various embodiments may be different, but do not have to be exclusive. For example, specific shapes, configurations, and characteristics of an embodiment may be used or implemented in another embodiment without departing from the inventive concepts.

Unless otherwise specified, the illustrated embodiments are to be understood as providing features of varying detail of some ways in which the inventive concepts may be implemented in practice. Therefore, unless otherwise specified, the features, components, modules, layers, films, panels, regions, and/or aspects, etc. (hereinafter individually or collectively referred to as “elements”), of the various embodiments may be otherwise combined, separated, interchanged, and/or rearranged without departing from the inventive concepts.

The use of cross-hatching and/or shading in the accompanying drawings is generally provided to clarify boundaries between adjacent elements. As such, neither the presence nor the absence of cross-hatching or shading conveys or indicates any preference or requirement for particular materials, material properties, dimensions, proportions, commonalities between illustrated elements, and/or any other characteristic, attribute, property, etc., of the elements, unless specified. Further, in the accompanying drawings, the size and relative sizes of elements may be exaggerated for clarity and/or descriptive purposes. When an embodiment may be implemented differently, a specific process order may be performed differently from the described order. For example, two consecutively described processes may be performed substantially at the same time or performed in an order opposite to the described order. Also, like reference numerals denote like elements.

When an element, such as a layer, is referred to as being “on,” “connected to,” or “coupled to” another element or layer, it may be directly on, connected to, or coupled to the other element or layer or intervening elements or layers may be present. When, however, an element or layer is referred to as being “directly on,” “directly connected to,” or “directly coupled to” another element or layer, there are no intervening elements or layers present. To this end, the term “connected” may refer to physical, electrical, and/or fluid connection, with or without intervening elements. Further, the D1-axis, the D2-axis, and the D3-axis are not limited to three axes of a rectangular coordinate system, such as the x, y, and z-axes, and may be interpreted in a broader sense. For example, the D1-axis, the D2-axis, and the D3-axis may be perpendicular to one another, or may represent different directions that are not perpendicular to one another. For the purposes of this disclosure, “at least one of X, Y, and Z” and “at least one selected from the group consisting of X, Y, and Z” may be construed as X only, Y only, Z only, or any combination of two or more of X, Y, and Z, such as, for instance, XYZ, XYY, YZ, and ZZ. As used herein, the term “and/or” includes any and all combinations of one or more of the associated listed items.

Although the terms “first,” “second,” etc. may be used herein to describe various types of elements, these elements should not be limited by these terms. These terms are used to distinguish one element from another element. Thus, a first element discussed below could be termed a second element without departing from the teachings of the disclosure.

Spatially relative terms, such as “beneath,” “below,” “under,” “lower,” “above,” “upper,” “over,” “higher,” “side” (e.g., as in “sidewall”), and the like, may be used herein for descriptive purposes, and, thereby, to describe one elements relationship to another element(s) as illustrated in the drawings. Spatially relative terms are intended to encompass different orientations of an apparatus in use, operation, and/or manufacture in addition to the orientation depicted in the drawings. For example, if the apparatus in the drawings is turned over, elements described as “below” or “beneath” other elements or features would then be oriented “above” the other elements or features. Thus, the exemplary term “below” can encompass both an orientation of above and below. Furthermore, the apparatus may be otherwise oriented (e.g., rotated 90 degrees or at other orientations), and, as such, the spatially relative descriptors used herein interpreted accordingly.

The terminology used herein is for the purpose of describing particular embodiments and is not intended to be limiting. As used herein, the singular forms, “a,” “an,” and “the” are intended to include the plural forms as well, unless the context clearly indicates otherwise. Moreover, the terms “comprises,” “comprising,” “includes,” and/or “including,” when used in this specification, specify the presence of stated features, integers, steps, operations, elements, components, and/or groups thereof, but do not preclude the presence or addition of one or more other features, integers, steps, operations, elements, components, and/or groups thereof. It is also noted that, as used herein, the terms “substantially,” “about,” and other similar terms, are used as terms of approximation and not as terms of degree, and, as such, are utilized to account for inherent deviations in measured, calculated, and/or provided values that would be recognized by one of ordinary skill in the art.

Various embodiments are described herein with reference to sectional and/or exploded illustrations that are schematic illustrations of idealized embodiments and/or intermediate structures. As such, variations from the shapes of the illustrations as a result, for example, of manufacturing techniques and/or tolerances, are to be expected. Thus, embodiments disclosed herein should not necessarily be construed as limited to the particular illustrated shapes of regions, but are to include deviations in shapes that result from, for instance, manufacturing. In this manner, regions illustrated in the drawings may be schematic in nature and the shapes of these regions may not reflect actual shapes of regions of a device and, as such, are not necessarily intended to be limiting.

As customary in the field, some embodiments are described and illustrated in the accompanying drawings in terms of functional blocks, units, and/or modules. Those skilled in the art will appreciate that these blocks, units, and/or modules are physically implemented by electronic (or optical) circuits, such as logic circuits, discrete components, microprocessors, hard-wired circuits, memory elements, wiring connections, and the like, which may be formed using semiconductor-based fabrication techniques or other manufacturing technologies. In the case of the blocks, units, and/or modules being implemented by microprocessors or other similar hardware, they may be programmed and controlled using software (e.g., microcode) to perform various functions discussed herein and may optionally be driven by firmware and/or software. It is also contemplated that each block, unit, and/or module may be implemented by dedicated hardware, or as a combination of dedicated hardware to perform some functions and a processor (e.g., one or more programmed microprocessors and associated circuitry) to perform other functions. Also, each block, unit, and/or module of some embodiments may be physically separated into two or more interacting and discrete blocks, units, and/or modules without departing from the scope of the inventive concepts. Further, the blocks, units, and/or modules of some embodiments may be physically combined into more complex blocks, units, and/or modules without departing from the scope of the inventive concepts.

As used throughout this disclosure, the expression ‘configured to’ (or ‘set to’) may be used interchangeably with, for example, ‘suitable for,’ ‘having the capacity to,’ ‘designed to,’ ‘adapted to,’ ‘made to,’ or ‘capable of,’ depending on the context. The term ‘configured to’ (or ‘set to’) may not necessarily mean only ‘specifically designed to’ in hardware. Instead, in some contexts, the expression ‘a system configured to’ may mean that the system is ‘capable of’ performing functions in conjunction with other devices or components. For example, the phrase ‘a processor configured to (or set to) perform A, B, and C’ may mean a dedicated processor (e.g., an embedded processor) for performing the operations, or a generic-purpose processor (e.g., a CPU or application processor) capable of performing the operations by executing one or more software programs stored in memory.

Unless otherwise defined, all terms (including technical and scientific terms) used herein have the same meaning as commonly understood by one of ordinary skill in the art to which this disclosure is a part. Terms, such as those defined in commonly used dictionaries, should be interpreted as having a meaning that is consistent with their meaning in the context of the relevant art and should not be interpreted in an idealized or overly formal sense, unless expressly so defined herein.

Functions related to artificial intelligence according to the present disclosure may be operated through a processor and memory. The processor may comprise one or a plurality of processors. In this case, the one or plurality of processors may be a generic-purpose processor such as a CPU, AP, or digital signal processor (DSP), a graphics-dedicated processor such as a GPU or vision processing unit (VPU), or an artificial intelligence-dedicated processor such as an NPU. The one or plurality of processors control processing of input data according to predefined operation rules or an artificial intelligence model stored in the memory. Alternatively, when the one or plurality of processors are an artificial intelligence-dedicated processors, the artificial intelligence-dedicated processors may be designed with a hardware structure specialized for processing a specific artificial intelligence model.

Artificial intelligence may be a field of computer engineering and information technology that studies methods for enabling computers to perform thinking, learning, self-development, and the like that may be performed by human intelligence, meaning that computers may imitate human intelligent behavior. In addition, artificial intelligence does not exist by itself, but has many direct and indirect relationships with other fields of computer science. In particular, in modern times, attempts have been very actively made to introduce artificial intelligence elements in various fields of information technology and utilize them for problem solving in those fields.

Machine learning may be a field of artificial intelligence that studies how to provide a computer with the ability to learn without explicit programming. Specifically, machine learning may be defined as a technology for studying and constructing systems that learn based on empirical data, perform prediction, and improve their own performance, along with algorithms therefor. Machine learning algorithms may take an approach of building specific models to derive predictions or decisions based on input data, rather than executing strictly predetermined static program instructions. The term ‘machine learning’ may be interchangeably used with the term ‘mechanical learning’.

Many machine learning algorithms may be developed regarding how to classify data in machine learning. Representative examples of such machine learning algorithms may include decision trees, Bayesian networks, support vector machines (SVM), and artificial neural networks (ANN).

A decision tree may be an analysis method that performs classification and prediction by plotting decision rules in a tree structure. A Bayesian network may be a model that expresses probabilistic relationships (conditional independence) between multiple variables in a graph structure. The Bayesian network is suitable for data mining through unsupervised learning.

The support vector machine is a model of supervised learning for pattern recognition and data analysis, and is mainly used for classification and regression analysis. An artificial neural network is an information processing system in which a plurality of neurons, called nodes or processing elements, are connected in the form of a layer structure by modeling the operation principle of biological neurons and the connection relationships between neurons.

The artificial neural network is a model used in machine learning, and is a statistical learning algorithm inspired by biological neural networks in biology (particularly the brain in the central nervous system of animals) in machine learning and cognitive science. Specifically, an artificial neural network may refer to an overall model in which artificial neurons (nodes) that form a network through connections of synapses change the synaptic connection strengths through learning to acquire problem-solving capabilities. The term artificial neural network may be used interchangeably with term neural network.

The artificial neural network may comprise a plurality of layers, and each of the layers may comprise a plurality of neurons. The artificial neural network may also comprise synapses connecting neurons to neurons. The artificial neural network may generally be defined by the following three factors: a connection pattern between neurons of different layers, a learning process for updating weights of the connections, and an activation function that generates an output value from a weighted sum of inputs received from a previous layer.

The artificial neural network may comprise, but is not limited to, network models such as a deep neural network (DNN), a recurrent neural network (RNN), a bidirectional recurrent deep neural network (BRDNN), a multilayer perceptron (MLP), and a convolutional neural network (CNN). In the present disclosure, the term ‘layer’ may be used interchangeably with the term ‘tier.’

The artificial neural network may be classified into a single-layer neural network and a multi-layer neural network according to the number of layers. A typical single-layer neural network may comprise an input layer and an output layer. A typical multi-layer neural network may comprise an input layer, one or more hidden layers, and an output layer.

The input layer may be a layer that receives external data, wherein the number of neurons in the input layer is equal to the number of input variables, the hidden layer is located between the input layer and the output layer and receives signals from the input layer, extracts features, and transmits the features to the output layer. The output layer may receive signals from the hidden layer and outputs an output value based on the received signals. The input signals between neurons may be multiplied by respective connection strengths (weights) and then summed, and when the sum is greater than a threshold of the neuron, the neuron may be activated to output an output value obtained through an activation function.

Meanwhile, the deep neural network including a plurality of hidden layers between the input layer and the output layer may be a representative artificial neural network that implements deep learning, which is a type of machine learning technology. The term ‘deep learning’ may be used interchangeably with the term ‘deep learning study’ in this disclosure.

A workflow of machine learning may include a series of processes of collecting data for learning and validation, and training a model after modeling, and may include processes of collecting training data, inspecting and exploring data, preprocessing and refining data, and modeling and training.

### 1. Collecting Training Data

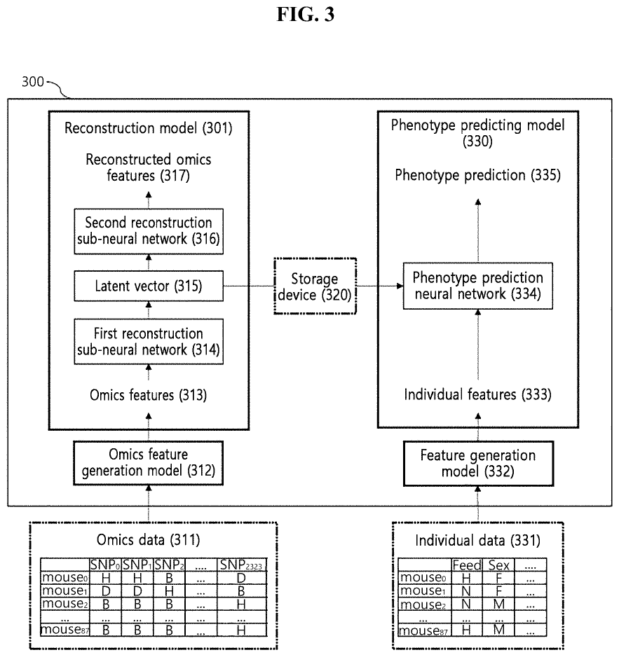

The system for predicting phenotypes of the present disclosure may acquire at least one or more omics data 311 (as shown in FIG. 3) and utilize the omics data 311 configured to learn phenotype prediction. The omics data 311 may refer to data used to understand biological phenomena by analyzing biomolecules such as genomics, transcriptomics, proteomics, metabolomics, and liposome.

The omics data 311 used in the system of the present disclosure may not be particularly limited, but as one example, omics data 311 selected from genomic data, transcriptomic data, and combinations thereof may be used. The genomic data that may be used in the system of the present disclosure may include DNA sequence data, and mutation data of DNA sequences may also be used. The mutations of DNA sequences may appear in the form of substitution, insertion, or deletion, and the mutation data of DNA sequences may not be particularly limited as long as data includes mutation information of DNA sequences, and may include mutation information resulting from substitution, insertion, and deletion of DNA sequences, and may include, for example, one or more selected from single nucleotide polymorphism (SNP), insertion-deletion (Indel), recombination, and combinations thereof.

The system for predicting phenotypes of the present disclosure may acquire one or more individual data 331 (as shown in FIG. 3) and utilize the individual data for learning phenotype prediction. In the present disclosure, the individual data may refer to data of the same individual as the individual constituting the acquired omics data for learning, and the type thereof is not particularly limited as long as the data may be utilized for phenotype prediction. For example, the individual data may include information such as dietary data or gender data of the individual.

Phenotype data may be used in learning the system for predicting phenotypes of the present disclosure, and may be used to compare the similarity between the phenotype predicted by the system for predicting phenotypes and an actual phenotype. Here, the phenotype may refer to an actual trait that appears according to genetic information as a result of genotype expression, and may be a concept that includes not only physical traits but also characteristics such as behaviors. The phenotype that is predicted in the present disclosure may not be particularly limited, but may be a disease-related phenotype. The disease-related phenotype may refer to a manifestation that is observable in an individual having a disease, and may include various aspects such as symptoms, changes in appearance, behaviors, and biochemical characteristics. For example, memory loss that appears in an individual having dementia may be a disease-related phenotype.

In the present disclosure, different types of training data sets may be used for learning, and each training data may further include one or more experiment-based results used as functional labels. At least a portion of the training data set may be used to train a learning model, and another portion may be used to evaluate performance of the trained learning model.

### 2. Inspecting and Exploring Training Data

Once training data for learning of the learning model is collected, the collected training data may be subjected to inspection and exploration for data structure, noise data, and data refinement methods for applying machine learning.

This step configured to inspect and explore the data may be called an exploratory data analysis (EDA) step, and EDA may be described as a process of observing and understanding collected data from various perspectives. Before learning the data, independent variables, dependent variables, variable types, data types of variables, and the like may be inspected through visualization such as graphs and statistical tests, and characteristics of the data and inherent structural relationships may be confirmed in advance. Through such EDA, phenomena represented by the data may be better understood and potential problems of the data may be identified by reviewing the distribution and values of the data. In addition, various patterns that cannot be identified in the problem definition step may be discovered through the process of inspecting the data from various perspectives, and based on this, existing hypotheses may be modified or new hypotheses may be established. The exploratory data analysis may broadly comprise a process of exploring outliers of the data and a process of analyzing relationships between data attributes.

The process of exploring outliers may refer to confirming whether outliers exist in the data, and may comprise sampling method, statistical method, visualization method, and the like. The sampling method may extract random samples from the data to check overall trends and peculiarities of the data values. The statistical method may use summary statistics such as mean, median, and mode that confirm a center of the data, or range and variance that confirm the dispersion of the data. The visualization method may determine which statistical indicators are appropriate for individual attributes of the collected data by utilizing probability density function, histogram, dot plot, word cloud, time-series chart, map, and the like. However, when using statistical indicators, it should be noted that since all data values in the set are reflected in the mean, the value is affected if outliers exist, whereas since one value located in the center is used for the median, representative results may be obtained even in the presence of outliers.

The process of analyzing relationships between data attributes may refer to find combinations of attributes that have meaningful correlations within the data, and relationship analysis may be performed differently depending to attribute combinations between categorical variables (qualitative) attributes that may not be numerically displayed but may be arbitrarily quantified, and numeric variables (quantitative) attributes that may be quantified. A categorical-categorical relationships may display the number of values corresponding to each pair of attribute values using cross tables or mosaic plots, a numeric-categorical relationship may observe statistical values (mean, median, etc.) for each category or be visually expressed through box plots, and a numeric-numeric relationship may analyze an association between two attributes by correlation coefficients. It may be confirmed that a correlation coefficient of −1 indicates a negative correlation where two attributes change in opposite directions, 0 indicates no correlation, and 1 indicates a positive correlation where two attributes always change in the same direction. The relationship between two attributes having a correlation coefficient may also exhibit various aspects, which may be visually expressed using scatter plots.

### 3. Preprocessing of Training Data

The data that has completed inspection and exploration may undergo data preprocessing to process the data into a form suitable for a model for machine learning training. Data preprocessing may involve refining data and transforming the data into a form that the model may understand, and may generally include handling missing data, outlier removal, data scaling, categorical data encoding, feature selection and extraction, and data transformation. The detailed processes of data preprocessing may be performed selectively in whole or in part, and a separate machine learning model may also be used for data preprocessing.

Handling missing data may involve processing missing values when the missing values exist in the data, wherein the missing values may be displayed as Not a Number (NaN) or blank values, or may be deleted. As the missing values are filled or deleted within the data, the integrity of the data may be improved, and when the missing values are filled, mean values, median values, mode values, or the like may be used.

Outlier Removal may involve removing outliers that deviate from a general data pattern in the data. Since outliers may degrade model performance, outliers should be removed or replaced, and the outliers may be identified and corresponding rows or columns may be deleted or replaced with other values.

Data Scaling may be a process of adjusting the sizes of data, wherein through data scaling, the range of data may be adjusted and model performance may be improved or convergence speed may be enhanced. Through data scaling, features of data may be adjusted to similar ranges, and generally, standardization and normalization may be applied to data scaling.

Categorical data encoding may involve the conversion of categorical variables expressed as string or integer values that cannot be directly input to a model, into numeric form that may be input into the model. Generally, one-hot encoding or label encoding may be used to convert categorical variables into numeric form.

Feature selection and extraction may be intended to improve of model performance by selecting the most useful features for model learning or extracting new features, and through this process, model complexity may be reduced and overfitting may be prevented.

Data transformation may be intended to transform data to extract new information or to enable better model understanding, and may include tokenization of text data or preprocessing of image data. Through the data transformation, useful features may be extracted from original data or the data may be transformed into appropriate form to improve model performance.

Through the data preprocessing as described above, the effects of improving performance and ensuring stability of machine learning models may be achieved.

Meanwhile, when the collected protein amino acid sequence-based data has not been preprocessed as needed, tokenization, cleaning, and normalization may be performed according to the intended use of the data.

In order to allow a computer to understand and process text, the text should be properly converted into numerical values. Since the performance of natural language processing may vary greatly depending on how words are represented, many technologies for quantifying words have been proposed. Currently, a word embedding method in which each word is vectorized through artificial neural network learning may be most widely used, and such word embedding methods may also be applied to omics data.

### 4. Training of Artificial Neural Networks

The artificial neural network may learn using a training data set. Here, the learning may refer to a process of determining parameters of the artificial neural network by using learning data in order to achieve purposes such as classifying, performing regression analysis on, or clustering input data. Representative examples of parameters of the artificial neural network may include weights assigned to synapses and biases applied to neurons.

The artificial neural network learned by the training data may classify or cluster input data according to patterns of the input data.

The following describes learning methods of artificial neural networks. The learning methods of artificial neural networks may be broadly classified into supervised learning, unsupervised learning, semi-supervised learning, and reinforcement learning.

Supervised learning may be a method of machine learning for inferring a function from training data. Among the functions thus inferred, outputting continuous values may be referred to as regression, and predicting and outputting a class of an input vector is referred to as classification.

In supervised learning, the artificial neural network may learn with labels provided for the training data. Here, the label may refer to a correct answer (or result value) that the artificial neural network should infer when the training data is input to the artificial neural network. In the present disclosure, the correct answer (or result value) that the artificial neural network should infer when training data is input may be referred to as a label or labeling data. In addition, in the present disclosure, setting a label to training data for learning the artificial neural network may be referred to as labeling the labeling data in the training data. In this case, the training data and the label corresponding to the training data may constitute one training set and may be input to the artificial neural network in the form of a training set.

Meanwhile, training data may represent a plurality of features, and labeling the training data may mean that the features represented by the training data are labeled. In this case, the training data may represent features of an input object in the form of a vector. The artificial neural network may infer a function for the association relationship between the training data and the labeling data by using the training data and labeling data. The parameters of the artificial neural network may then be determined (optimized) through evaluation of the function inferred by the artificial neural network.

Unsupervised learning may be a type of machine learning in which no labels are provided for the training data. Specifically, unsupervised learning may be a learning method of training the artificial neural network to find and classify patterns in the training data itself, rather than focusing on the association relationship between training data and corresponding labels. Examples of unsupervised learning may include clustering and independent component analysis.

In the present disclosure, the term ‘clustering’ may be used interchangeably with the term ‘clustering’.

Examples of artificial neural networks using unsupervised learning may include generative adversarial network (GAN) and autoencoder (AE).

The generative adversarial network may be a machine learning method in which two different artificial intelligence models, a generator and a discriminator, compete to improve performance. In this case, the generator may be a model that creates new data and may generate new data based on original data. The discriminator may be a model that recognizes patterns of data and may perform a role of determining whether input data is original data or new data generated by the generator. The generator may receive as input and learn from data that failed to deceive the discriminator, while the discriminator may receive as input and learn from data that was successfully deceived by the generator. Accordingly, the generator may evolve to deceive the discriminator as well as possible, while the discriminator may evolve to well distinguish between original data and data generated by the generator.

An autoencoder may be a neural network that aims to reproduce the input itself as an output. The autoencoder may include an input layer, at least one hidden layer, and an output layer. In this case, since the number of nodes in the hidden layer is less than the number of nodes in the input layer, a dimension of the data may be reduced, and thus compression or encoding may be performed. In addition, data output from the hidden layer may enter the output layer. In this case, since the number of nodes in the output layer is greater than the number of nodes in the hidden layer, the dimension of the data may be increased, and thus decompression or decoding may be performed.

Meanwhile, the autoencoder may adjust the connection strength of neurons through learning so that input data is represented as hidden layer data. In the hidden layer, the fact that information is represented by fewer neurons than in the input layer, and input data may be reproduced as output may mean that the hidden layer discovers and represents hidden patterns from the input data.

Semi-supervised learning may be a type of machine learning, and may refer to a learning method using both labeled training data and unlabeled training data. As one technique of semi-supervised learning, there may be a technique of inferring labels of unlabeled training data and then performing learning using the inferred labels, and this technique may be usefully employed when labeling costs are high.

Reinforcement learning may be a theory that if an environment is given in which an agent may determine what action should be taken at each moment, the agent may find the best path through experience without data. Reinforcement learning may be mainly performed by a Markov decision process (MDP). To explain the Markov decision process, first, an environment may be given in which information required for the agent to take the next action is configured; second, how the agent act in that environment, may be defined; third, what the agent does well is rewarded and what it does poorly is penalized, may be defined; and fourth, the agent repeatedly may experience until future rewards reach their maximum to derive an optimal policy.

The structure of an artificial neural network may be specified by model configuration, activation function, loss function or cost function, learning algorithm, optimization algorithm, and the like, wherein hyperparameters are preset before learning, and thereafter model parameters may be set through learning to specify the content.

For example, elements determining the structure of the artificial neural network may include the number of hidden layers, the number of hidden nodes included in each hidden layer, an input feature vector, a target feature vector, and the like.

The hyperparameters may include various parameters that should be initially set for learning, such as initial values of model parameters. The model parameters may include various parameters to be determined through learning. For example, the hyperparameters may include initial values of inter-node weights, initial values of inter-node bias, mini-batch size, number of learning iterations, learning rate, and the like. And the model parameters may include inter-node weights, inter-node bias, and the like.

The loss function may be used as an indicator (criterion) for determining optimal model parameters in the learning process of the artificial neural network. In artificial neural networks, learning refers to a process of manipulating model parameters to reduce the loss function, and the purpose of learning may be regarded as determining model parameters that minimize the loss function. The loss function may mainly use mean squared error (MSE) or cross entropy error (CEE), but the present disclosure is not limited thereto. The cross entropy error may be used when correct answer labels are one-hot encoded. The one-hot encoding is an encoding method in which a correct answer label value is set to 1 only for neurons corresponding to correct answers, and correct answer label values are set to 0 for non-correct neurons.

In machine learning or deep learning, a learning optimization algorithm may be used to minimize a loss function, and the learning optimization algorithms may include gradient descent (GD), stochastic gradient descent (SGD), momentum, Nesterov accelerated gradient (NAG), Adagrad, AdaDelta, RMSProp, Adam, Nadam, and the like.

Gradient descent may be a technique configured to adjust model parameters in a direction that reduces the loss function value by considering the gradient of the loss function in the current state. The direction in which the model parameters are adjusted may be referred to as the step direction, and the magnitude of the adjustment may be referred to as the step size. In this case, the step size may refer to a learning rate. In gradient descent, the gradient may be obtained by partially differentiating the loss function with respect to each model parameter, and the model parameters may be updated by changing them in the direction of the obtained gradient by an amount equal to the learning rate.

Stochastic gradient descent may be a technique that divides learning data into mini-batches and perform gradient descent for each mini-batch to increase the frequency of gradient descent.

Adagrad, AdaDelta, and RMSProp may be techniques configured to adjust step size in SGD to improve optimization accuracy. Momentum and NAG in SGD are techniques configured to adjust step direction to improve optimization accuracy. Adam may be a technique for combining momentum and RMSProp to adjust both step size and step direction to improve optimization accuracy. Nadam may be a technique for combining NAG and RMSProp to adjust both step size and step direction to improve optimization accuracy.

The learning speed and accuracy of artificial neural networks may be characterized by being greatly dependent not only on the structure of the artificial neural network and the type of learning optimization algorithm, but also on hyperparameters. Therefore, in order to obtain a good learning model, it is important not only to determine an appropriate structure of the artificial neural network and the learning algorithm, but also to set appropriate hyperparameters.

Typically, hyperparameters may be experimentally set to various values while learning the artificial neural network, and are set to optimal values that provide stable learning speed and accuracy based on the learning results.

The system configured to predict phenotypes of the present disclosure may learn to predict phenotypes from omics data of an individual by using a training dataset including omics data.

FIG. 3 is a schematic example diagram showing learning steps of the system for predicting phenotypes of the present disclosure. Referring to FIG. 3, the system configured to predict phenotypes 300 of the present disclosure may include a reconstruction model 310 and a phenotype predicting model 330, wherein the reconstruction model receiving the omics feature 313 as input generates a latent vector 315, and the phenotype predicting model receiving the latent vector as input predicts a disease-associated phenotype 335.

The reconstruction model of the system configured to predict phenotypes of the present disclosure performs a function of identifying significant features from omics features. While the biological information included in omics data is vast, much of this biological information may be irrelevant to a specific phenotype, such that learning efficiency is significantly degraded when learning is performed using omics data having vast biological information as is. In the system for predicting phenotypes of the present disclosure, the reconstruction model learns to extract only significant features from omics features, and by utilizing the learned reconstruction model for phenotype prediction, the efficiency and prediction reliability of the system for predicting phenotypes may be enhanced.

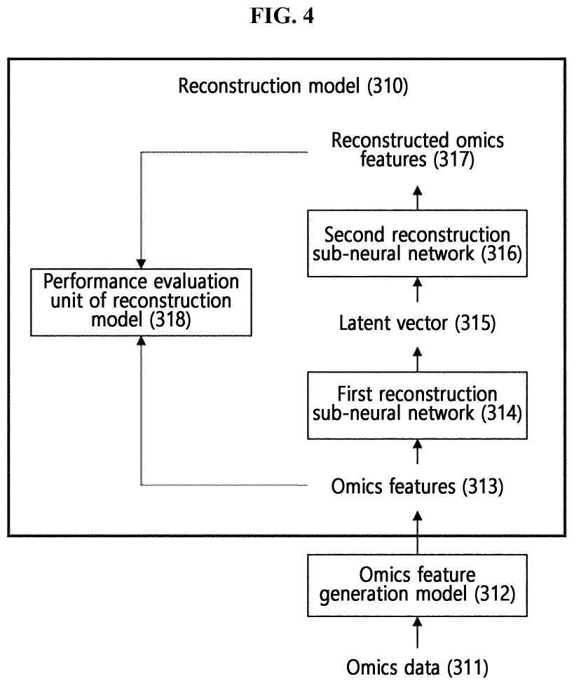

FIG. 4 is a schematic example diagram showing a learning process of a reconstruction model included in the system for predicting phenotypes of the present disclosure.

Referring to FIG. 4, the reconstruction model 310 may include a first reconstruction sub-neural network 314 and a second reconstruction sub-neural network 316. The first reconstruction sub-neural network may compress a dimension of the input omics feature 313 to generate a latent vector 315. The second reconstruction sub-neural network may expand the compressed latent vector to generate a reconstructed omics feature 317.

The reconstruction model may further include a performance evaluation unit 318 of the reconstruction model, and the performance evaluation unit of the reconstruction model may receive as input an omics feature 313 input to the reconstruction model and a reconstructed omics feature 317 generated using the reconstruction model, evaluate whether the reconstruction model have reconstructed the omics feature similarly to the input omics feature, and output an evaluation result. The reconstruction model may receive the value output from the performance evaluation unit of the reconstruction model as input and may learn to reconstruct the omics features similarly to the input omics features.

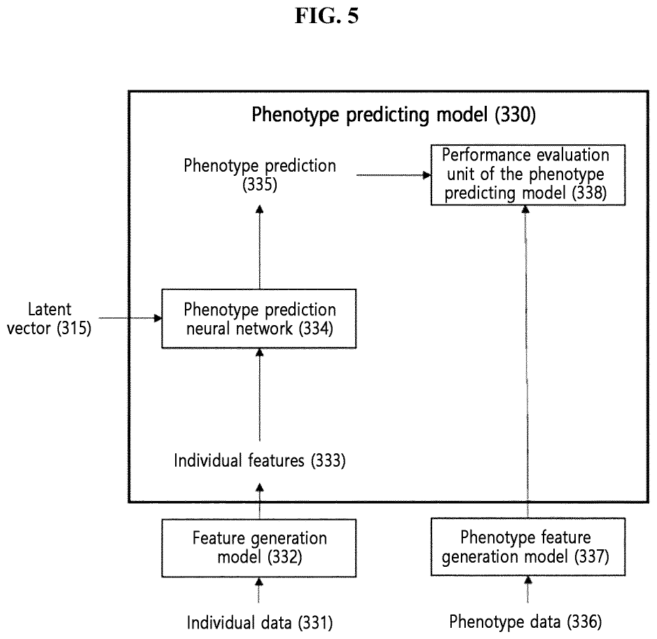

FIG. 5 is a schematic example diagram showing a learning process of a phenotype predicting model included in the system for predicting phenotypes of the present disclosure.

Referring to FIG. 5, the phenotype predicting model may include a phenotype prediction neural network 334. The phenotype prediction neural network may receive the latent vector 315 generated by the reconstruction model as input and predict the phenotype 335. In addition, the phenotype prediction neural network may further receive individual features 333 generated from individual data 331 as input and predict the phenotype 335. By additionally utilizing the individual data for learning, the efficiency and performance of the system for predicting phenotypes may be further improved.

The phenotype predicting model may further include a performance evaluation unit 337 of the phenotype predicting model, and may further receive phenotype features as input for evaluating performance of the phenotype predicting model. The performance evaluation unit of the phenotype predicting model may evaluate whether the phenotype prediction neural network have predicted the phenotype similarly to the phenotype data 336 and outputs an evaluation result. The system for predicting phenotypes of the present disclosure may receive the value output from the performance evaluation unit of the phenotype predicting model and learn to predict the phenotype 335 similarly to the phenotype data 336.

The system for predicting phenotypes of the present disclosure may learn at least one or more. An order of learning of the reconstruction model and the phenotype predicting model may not be particularly limited, but the reconstruction model and the phenotype predicting model may learn separately, the reconstruction model and phenotype predicting model may learn alternately, or the phenotype predicting model may further learn after the reconstruction model and phenotype predicting model have learned alternately.

In an embodiment of the present disclosure, after the reconstruction model has completed learning, the latent vector generated by the learned reconstruction model may be stored in the storage device 320, and the phenotype predicting model that receives the stored latent vector as input, may perform learning, whereby the reconstruction model and the phenotype predicting model may learn respectively.

In another embodiment, after the phenotype predicting model performs learning by receiving as input the latent vector generated during the learning process of the reconstruction model, the phenotype predicting model may perform learning again by receiving as input the latent vector generated by the learned reconstruction model, whereby the reconstruction model and the phenotype predicting model may learn alternately.

In yet another embodiment, after the phenotype predicting model performs learning by receiving as input the latent vector generated during the learning process of the reconstruction model, the phenotype predicting model may perform learning again by receiving as input the latent vector generated by the learned reconstruction model, whereby the reconstruction model and the phenotype predicting model learn alternately, and after that, the latent vector generated by the learned reconstruction model may be stored in a storage device, and the phenotype predicting model that receives the stored latent vector as input may further perform learning.

### Mixture of Experts (MoE) Architecture

In an embodiment of the present disclosure, a system for predicting phenotypes may be implemented utilizing a model architecture such as MoE. Here, the MoE may refer to an architecture of a machine learning model that solves complex problems by combining multiple expert models.

Such MoE may include expert models, which are multiple small networks designed to learn different portions and/or different features of predetermined data and perform corresponding data processing operations, and a gating network, which evaluates the performance of each expert model and determines, based thereon, which expert model is most suitable for assigning a specific task according to the predetermined data.

Thus, according to the MoE architecture, the gating network that acquires predetermined input data may determine probabilistic or deterministic task assignment for each expert model, and the selected expert models perform their respective tasks and return the results, thereby performing data processing for a specific task.

The MoE model used according to an embodiment of the present disclosure may refer to a predetermined MoE model implemented according to conventional methods known in the art. By way of example, the MoE model may include Switch Transformer, Conditional Computation in Neural Networks, Sparse Mixture of Experts, and/or Megatron-LM.

In addition, in an embodiment of the present disclosure, the system may include an MoE model based on a combination of a plurality of specialized models (SM) and a router (Gating Network, RT), and may also include a domain-specific specialized model-based MoE model.

By utilizing such MoE, the overall efficiency and performance of the system for predicting phenotypes of the present disclosure may be enhanced by concentrating computational resources through activation of only specific portions when handling complex tasks or large datasets.

### 5. Computing System Apparatus

An embodiment of the present disclosure may be implemented as an application specific integrated circuit (ASIC) that is manufactured to suit specific application fields and specialized functions of devices.

The application-specific integrated circuit may also be called an application-specific semiconductor, and unlike standard semiconductors that have established specifications and may be applied to any electronic product or application as long as certain requirements are met, the application-specific semiconductor is an integrated circuit that is used for a specific product or function and is manufactured by a semiconductor manufacturer according to specific orders. In particular, application-specific semiconductor may be designed and manufactured to perform only the functions necessary for a specific device or specific function, and the application-specific semiconductor may be broadly divided according to design methodology into full custom IC that designs and manufactures a circuit from the beginning according to user requirements, and semi-custom IC that designs and manufactures a circuit by partially using standardized designs.

The application-specific semiconductor may be mainly used in communication systems, high-performance computing systems, consumer electronic products, automobiles, industrial automation, medical devices, military, aerospace industries, and the like, and have recently been applied to AI semiconductors that execute large-scale computations required for AI implementation with high performance and power efficiency.

In communication systems, the application-specific semiconductor may be used as key components for network routers, switches, and modems, and perform data packet processing, protocol conversion, signal processing, and the like, thereby providing high throughput and low latency. In high-performance computing systems, the ASIC may be used as key components for high-speed processing and parallel processing, and in consumer electronics such as digital cameras, smartphones, tablets, and game consoles, the ASIC may provide high-performance and low-power solutions necessary for performing specific functions. In the automobile industry, the ASIC may be used to control various electronic systems inside the automobiles, and in industrial automation systems, the ASIC may provide solutions for high-precision control and high-performance processing.

The application-specific integrated circuit to which an embodiment of the present disclosure may be applied may include a memory in which individual memory interface (I/F) is implemented, and may include a plurality of functional blocks that request memory access. Each functional block may be a direct memory access (DMA) functional block, a processor, a video processor, a cache controller, a decompression block, or a data path block. A basic configuration of the application-specific integrated circuit may include transistors that amplify or switch electrical signals, logic gates that are circuits performing logical functions by combining transistors, memory cells that store data, analog circuits that are circuits for processing continuous voltage or current by combining transistors, and IP cores (Intellectual Property Cores) such as a microprocessor, a DSP, or a graphic core which are pre-designed to perform specific functions.

The ASIC may also include an individual memory I/F for interfacing with the individual memory and an embedded memory I/F for interfacing with the embedded memory, wherein the individual memory I/F may be coupled to each functional block to receive memory access signals (e.g., control signals, address signals, and data signals) and generate signals for controlling the individual memory based on these input signals. The embedded memory I/F is coupled to each functional block to receive memory access signals (e.g., control signals, address signals, data signals) and to generate modified memory access signals for controlling the embedded memory based on these input signals. The individual memory I/F and the embedded memory I/F may be designed within the memory control block of the ASIC to provide a memory control structure that may be flexibly applied to both the individual memory and the embedded memory.

In addition, an application-specific integrated circuit (ASIC) for an artificial neural network may comprise a plurality of neurons configured in an array and a plurality of synaptic circuits, wherein each neuron may comprise a register, a microprocessor, and at least one input, and each synaptic circuit may comprise a memory for storing a synaptic weight. Here, each neuron of the ASIC may be connected to at least one other neuron through one of the plurality of synaptic circuits.

While the present disclosure is generally described above as being implementable by a computing apparatus, those skilled in the art will recognize that the present disclosure may be implemented in combination with computer-executable instructions and/or other program modules that may be executed on one or more computers and/or as a combination of hardware and software.

Those having ordinary skill in the art will understand that information and signals may be represented using any of a variety of different technologies and techniques. For example, data, instructions, commands, information, signals, bits, symbols, and chips that may be referenced in the above description may be represented by voltages, currents, electromagnetic waves, magnetic fields or particles, optical fields or particles, or any combination thereof.

Those having ordinary skill in the art will understand that the various illustrative logical blocks, modules, processors, means, circuits, and algorithm steps described in connection with the embodiments disclosed herein may be implemented as electronic hardware, various forms of program or design code (referred to herein as ‘software’ for convenience), or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Those having ordinary skill in the art may implement the described functionality in various ways for each particular application, but such implementation decisions should not be interpreted as departing from the scope of the present disclosure.

The various embodiments presented herein may be implemented as a method, apparatus, or article of manufacture using standard programming and/or engineering techniques. The term ‘article of manufacture’ may include a computer program, carrier, or medium accessible from any computer-readable storage device. For example, computer-readable storage medium may include, but is not limited to, magnetic storage devices (e.g., hard disk, floppy disk, magnetic strips, etc.), optical disks (e.g., CD, DVD, etc.), smart cards, and flash memory devices (e.g., EEPROM, card, stick, key drive, etc.). In addition, the various storage media presented herein include one or more devices and/or other machine-readable media for storing information.

It should be understood that the specific order or hierarchy of steps in the processes presented is an example of exemplary approaches. Based on design priorities, it is understood that the specific order or hierarchy of steps in the processes may be rearranged within the scope of the present disclosure. The accompanying method claims present elements of the various steps in a sample order, and are not meant to be limited to the specific order or hierarchy presented.

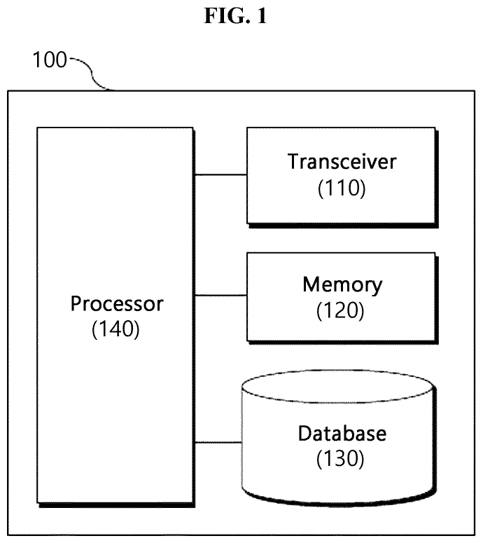

FIG. 1 illustrates a schematic example block diagram of a computing system apparatus 100 that implements a phenotype prediction service according to an embodiment of the present disclosure.

The various operations of the system for predicting phenotypes and method of the present disclosure may be performed by any suitable means capable of performing the corresponding functions. Such means may include various hardware, software components, modules, and combinations thereof, including, but not limited to, circuits, processors, application specific integrated circuits (ASICs), and field programmable gate arrays (FPGAs).

Referring to FIG. 1, a computing system 100 implementing an artificial neural network of the present disclosure may include a transceiver 110, a memory 120, a database 130, and a processor 140. However, not all components shown in FIG. 1 are essential components of the computing system 100. The computing system 100 may be implemented with more components than those shown in FIG. 1, or may be implemented with fewer components than those shown in FIG. 1. Furthermore, the transceiver 110, the memory 120, and the processor 140 may be implemented in the form of a single chip.

In one embodiment, the transceiver 110 may communicate with a terminal connected to the computing system apparatus 100 by wire or wirelessly, or with another electronic device. For example, the transceiver 110 may obtain protein amino acid sequence information, protein structure information, or a protein representation generated using an artificial neural network from another electronic device.

Various types of data such as programs including applications and files may be installed and stored in the memory 110. The processor 140 may access and use the data stored in the memory 110, or may store new data in the memory 110. In addition, one or more instructions may be stored in the memory 110. The processor 140 may execute the one or more instructions stored in the memory.

The processor 140 may control the overall operation of the computing system apparatus 100. Here, the processor 140 may be configured as at least one of a central processing unit (CPU), a graphics processing unit (GPU), an application-specific integrated circuit, a digital signal processor (DSP), a digital signal processing device (DSPD), a programmable logic device (PLD), a field programmable gate array, controllers, micro-controllers, microprocessors, and/or other electrical units for performing other functions, or a plurality of electrically connected processors.

The processor 140 may control other components included in the computing system apparatus 100 to perform operations for operating the computing system apparatus 100. For example, the processor 140 may obtain protein amino acid sequence information, utilize the neural network to obtain a protein expression, calculate a contrastive loss function from the protein representations, and improve one or more numerical values or parameters of one or more neural networks among the encoder neural network and the projection neural network based on the contrastive loss function.

The database 130 may store various learning data for training the learning model. In addition, the database 130 may store protein amino acid sequence information, protein structure information, simulation result information, and the like, and in various embodiments, output data generated by the learning model may also be stored. Although the computing system apparatus 100 is illustrated as including the database 130 in FIG. 1, the database 130 may also be provided outside the apparatus. In this case, the database 130 may be connected to the computing system apparatus 100 in a wired or wireless manner.

In addition, the learning model of the present disclosure may be implemented external to the computing system apparatus 100 (e.g., implemented in a cloud-based manner) or may be included within the computing system apparatus 100.

An embodiment of the present disclosure may also be implemented in the form of a recording medium including computer-executable instructions, such as program modules executed by a computer. The computer-readable medium may be any available medium accessible by a computer, and may include both volatile and non-volatile media, removable and non-removable media. In addition, the computer-readable medium may include both computer storage media and communication media. The computer storage media may include both volatile and non-volatile, removable and non-removable media implemented in any method or technology for storage of information such as computer-readable instructions, data structures, program modules, or other data. The communication media typically may include computer-readable instructions, data structures, or program modules, and may include any information delivery media.

Throughout the present disclosure, the device may include, but is not limited to, a server, a smartphone, a tablet PC, a PC, a TV, a smart TV, a mobile phone, a personal digital assistant (PDA), a speaker, a laptop, a media player, a microserver, an electronic book object recognition device, an object recognition device for digital broadcasting, a kiosk, an MP3 player, a digital camera, a robot vacuum cleaner, an home appliance, other mobile or non-mobile computing devices, a watch, glasses, a hair band, a ring, and the like having communication functions and data processing functions.

### 6. Application of System for Predicting Phenotypes

The system for predicting phenotypes of the present disclosure may be used for various purposes, such as providing information for diagnosing a disease in an individual, assessing a risk for a disease in an individual, or searching for biomolecules associated with a disease.

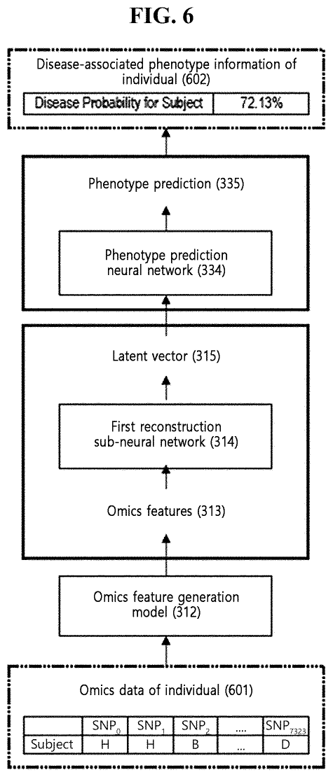

FIG. 6 is a schematic example diagram showing steps for predicting disease-associated phenotypes using the system for predicting phenotypes of the present disclosure. The system for predicting phenotypes that receives omics data 601 of an individual as input may generate disease-associated phenotype information 602 of the individual through a reconstruction model and a phenotype predicting model, and the disease-associated phenotype information 602 of an individual generated by the system for predicting phenotypes of the present disclosure may be used to accurately diagnose diseases of the individual or to evaluate a risk of the diseases of the individual.

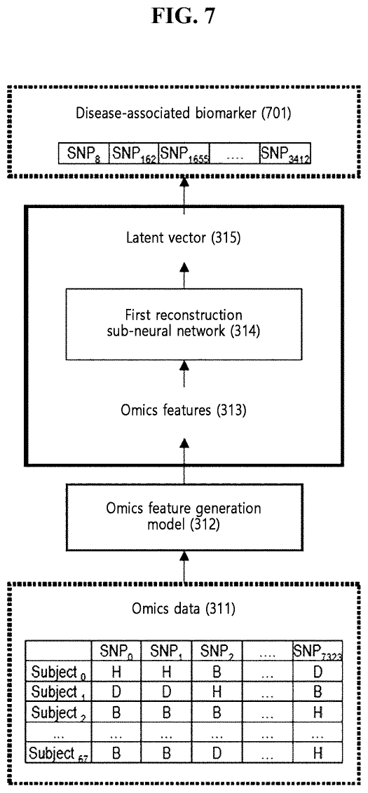

FIG. 7 is a schematic example diagram showing steps of searching for disease-associated biomolecules using the system for predicting phenotypes of the present disclosure. The system for predicting phenotypes that receives the omics data 301 as input may generate a latent vector 315 through a reconstruction model, and may obtain disease-associated biomarker 701 information from the latent vector. Such disease-associated biomarker 701 information may be utilized for discovering novel targets for new drug development.

The foregoing description of the present disclosure is for illustrative purposes, and it will be understood by those skilled in the art to which the invention pertains that other specific forms may be easily modified without changing the technical spirit or essential features of the invention. For example, each component described as a single unit may be implemented in a distributed manner, and likewise, the components described as distributed may be implemented in a combined form.

The scope of the present disclosure is indicated by the claims set forth below rather than by the detailed description above, and all changes or modifications derived from the meaning and scope of the claims and their equivalent concepts should be construed as being included within the scope of the present disclosure.

Hereinafter, the present disclosure will be described in more detail by way of examples. The examples of the present disclosure are illustrative in all respects, and the present disclosure is not limited to these examples only.

### Example 1

Omics features may be generated from the omics data of the AD-BXD panel, and after the reconstruction model completed learning using the generated omics features, latent vectors may be generated from the omics features using the reconstruction model and stored in a storage device. Thereafter, the phenotype predicting model learned to predict contextual fear acquisition (CFA) and contextual fear memory (CFM) by utilizing the stored latent vectors as input values.

### Example 2

After the reconstruction model and the phenotype predicting model learned alternately, latent vectors may be generated from the omics features using the reconstruction model and stored in a storage device. The phenotype predicting model learned to predict contextual fear acquisition and contextual fear memory by utilizing the stored latent vectors as input values.

### Example 3

The reconstruction model and the phenotype predicting model learned alternately so as to predict fear memory acquisition and fear memory recall.

### Comparative Example 1

Omics features may be generated from the omics data of the AD-BXD panel, and the generated omics features may be used as input values to learn the system for predicting phenotypes.

### Performance Evaluation of System for Predicting Phenotypes

The performance of the system for predicting phenotypes of the examples and comparative examples of the present disclosure may be evaluated by root mean squared error (RMSE) and cross-validation may be performed using Pearson correlation coefficient. A lower root mean squared error may indicate superior performance of the system for predicting phenotypes, and a higher Pearson correlation coefficient may indicate superior performance of the system for predicting phenotypes.

TABLE 1

| | fear memory acquisition prediction performance | |
|---|---|---|
| | root mean square error | Pearson correlation coefficient |
| Example 1 | 0.1420 | 0.4404 |
| Example 2 | 0.1376 | 0.4648 |
| Example 3 | 0.1362 | 0.4713 |
| Comparative | 0.1452 | 0.4205 |
| Examples | | |

TABLE 2

| | fear memory recall prediction performance | |
|---|---|---|
| | root mean square error | Pearson correlation coefficient |
| Example 1 | 0.1492 | 0.5788 |
| Example 2 | 0.1364 | 0.6404 |
| Example 3 | 0.1356 | 0.6431 |
| Comparative | 0.1514 | 0.5709 |
| Example 1 | | |

The system for predicting phenotypes of Examples 1, 2, and 3 exhibited lower root mean square error values and higher Pearson correlation coefficient values in predicting fear memory acquisition and fear memory recall, as compared to the system for predicting phenotypes without the reconstruction model (Comparative Example) (Tables 1 and 2). The above results may demonstrate that the system for predicting phenotypes of the present disclosure exhibits superior phenotype prediction performance, as compared to the system for predicting phenotypes of the comparative example.

The system and method for predicting phenotypes of the present disclosure exhibit excellent performance in phenotype prediction compared with conventional methods, and may perform learning more efficiently than conventional methods.

By using the system and method for predicting phenotypes of the present disclosure, more reliable prediction results may be efficiently obtained, and the system and method for predicting phenotypes of the present disclosure may be utilized to obtain information for diagnosis of a disease state of an individual, to obtain information for evaluating a disease risk of an individual, and also for target discovery for new drug development.

Although certain embodiments and implementations have been described herein, other embodiments and modifications will be apparent from this description. Accordingly, the inventive concepts are not limited to such embodiments, but rather to the broader scope of the appended claims and various obvious modifications and equivalent arrangements as would be apparent to a person having ordinary skill in the art.

What is claimed is:

1. A system for predicting phenotypes comprising:
at least one memory configured to store one or more instructions; and
at least one processor configured to execute the one or more instructions stored in the at least one memory,
wherein operations performed by the one or more instructions include learning to predict phenotypes from omics data, and
wherein the learning to predict phenotypes includes:
acquiring at least one dataset including omics data;
generating omics features from the omics data;
generating, by a reconstruction model, a latent vector from the omics features; and
predicting, by a phenotype predicting model, phenotypes from the latent vector.

2. The system for predicting phenotypes of claim 1,
wherein the reconstruction model includes a first reconstruction sub-neural network and a second reconstruction sub-neural network, the first reconstruction sub-neural network generating the latent vector from the omics features, and the second reconstruction sub-neural network generating reconstructed omics features from the latent vector,
wherein the learning to predict phenotypes includes:
generating, by the first reconstruction sub-neural network, the latent vector from the omics features;
generating, by the second reconstruction sub-neural network, the reconstructed omics features from the latent vector;
comparing the reconstructed omics features with the omics features to evaluate performance of the reconstruction model; and
learning the reconstruction model based on the evaluation.

3. The system for predicting phenotypes of claim 2,
wherein the learning to predict phenotypes includes at least one of:
respectively learning the reconstruction model and the phenotype predicting model;
alternately learning the reconstruction model and the phenotype predicting model; or
after alternately learning the reconstruction model and the phenotype predicting model, additionally learning the phenotype predicting model.

4. The system for predicting phenotypes of claim 1,
wherein the phenotype predicting model further receives one or more individual features as input to predict phenotypes.

5. The system for predicting phenotypes of claim 1,
wherein the phenotypes are associated with a disease.

6. The system for predicting phenotypes of claim 1,
wherein the omics data includes one or more selected from genomic data, transcriptomic data, proteomic data, metabolomic data, lipidomic data, and combinations thereof.

7. The system for predicting phenotypes of claim 1, wherein the system is configured to:
obtain disease-associated biomarker information or disease-associated phenotype information of an individual.

8. A method for predicting phenotypes performed by at least one processor, the method comprising:
learning to predict phenotypes from omics data,
wherein the learning to predict phenotypes includes:
acquiring at least one dataset including omics data;
generating omics features from the omics data;
generating, by a reconstruction model, a latent vector from the omics features; and
predicting, by a phenotype predicting model, phenotypes from the latent vector.

9. The method for predicting phenotypes of claim 8,
wherein the reconstruction model includes a first reconstruction sub-neural network and a second reconstruction sub-neural network, the first reconstruction sub-neural network generating the latent vector from the omics features, and the second reconstruction sub-neural network generating reconstructed omics features from the latent vector, and
wherein the learning to predict phenotypes includes:
generating, by the first reconstruction sub-neural network, the latent vector from the omics features;
generating, by the second reconstruction sub-neural network, the reconstructed omics features from the latent vector;
comparing the reconstructed omics features with the input omics features to evaluate performance of the reconstruction model; and
learning the reconstruction model based on the evaluation.

10. The method for predicting phenotypes of claim 9,
wherein the learning to predict phenotypes includes at least one of:
respectively learning the reconstruction model and the phenotype predicting model;
alternately learning the reconstruction model and the phenotype predicting model; or
after alternately learning the reconstruction model and the phenotype predicting model, additionally learning the phenotype predicting model.

11. The method for predicting phenotypes of claim 8,
wherein the phenotype predicting model further receives one or more individual features as input to predict phenotypes.

12. The method for predicting phenotypes of claim 8,
wherein the phenotypes are associated with a disease.

13. The method for predicting phenotypes of claim 8,
wherein the omics data includes one or more selected from genomic data, transcriptomic data, proteomic data, metabolomic data, lipidomic data, and combinations thereof.

14. The method for predicting phenotypes of claim 8, further comprising:
obtaining disease-associated biomarker information or obtaining disease-associated phenotype information of an individual.

15. A program stored on a computer-readable recording medium to execute a method for predicting phenotypes performed by at least one processor, the method comprising:
learning to predict phenotypes from omics data,
wherein the learning to predict phenotypes includes:
acquiring at least one dataset including omics data;
generating omics features from the omics data;
generating, by a reconstruction model, a latent vector from the omics features; and
predicting, by a phenotype predicting model, phenotypes from the latent vector.

16. A computerized method for predicting phenotypes, the method comprising:
learning to predict phenotypes from omics data,
wherein the learning to predict phenotypes includes:
acquiring at least one dataset including omics data;
generating omics features from the omics data;
generating, by a reconstruction model, a latent vector from the omics features; and
predicting, by a phenotype predicting model, phenotypes from the latent vector.

17. The method for predicting phenotypes of claim 16,
wherein the reconstruction model includes a first reconstruction sub-neural network and a second reconstruction sub-neural network, the first reconstruction sub-neural network generating the latent vector from the omics features, and the second reconstruction sub-neural network generating reconstructed omics features from the latent vector, and
wherein the learning to predict phenotypes includes:
generating, by the first reconstruction sub-neural network, the latent vector from the omics features;
generating, by the second reconstruction sub-neural network, the reconstructed omics features from the latent vector;
comparing the reconstructed omics features with the input omics features to evaluate performance of the reconstruction model; and
learning the reconstruction model based on the evaluation.

18. The method for predicting phenotypes of claim 17,
wherein the learning to predict phenotypes includes at least one of:
respectively learning the reconstruction model and the phenotype predicting model;
alternately learning the reconstruction model and the phenotype predicting model; or
after alternately learning the reconstruction model and the phenotype predicting model, additionally learning the phenotype predicting model.

19. The method for predicting phenotypes of claim 16,
wherein the phenotype predicting model further receives one or more individual features as input to predict phenotypes.

20. The method for predicting phenotypes of claim 16,
wherein the phenotypes are associated with a disease.

21. The method for predicting phenotypes of claim 16,
wherein the omics data includes one or more selected from genomic data, transcriptomic data, proteomic data, metabolomic data, lipidomic data, and combinations thereof.

22. The method for predicting phenotypes of claim 16, further comprising:
obtaining disease-associated biomarker information or obtaining disease-associated phenotype information of an individual.
